(19) United States
(12) Patent Application Publication
(10) Pub. No.: US 2021/0379268 A1
(43) Pub. Date: Dec. 9, 2021

(54) METHOD AND SYSTEMS FOR CLEARING BLOOD OF ACTIVE PATHOGENS

(71) Applicant: Levine Machine, L.L.C., Phoenix, AZ (US)

(72) Inventor: Michael Adam LEVINE, Phoenix, AZ (US)

(73) Assignee: Levine Machine, L.L.C., Phoenix, AZ (US)

(21) Appl. No.: 17/339,103

(22) Filed: Jun. 4, 2021

Related U.S. Application Data
(60) Provisional application No. 63/035,528, filed on Jun. 5, 2020.

(51) Int. Cl. A61M 1/36; A61K 35/14

(52) U.S. Cl. CPC A61M 1/3683; A61K 35/14; A61M 2202/30; A61M 2205/702; A61M 2205/3584; A61M 2205/3553; A61M 2202/20

(57) ABSTRACT

A system, device, and autologous method for treating blood. Blood is extracted from a patient. The blood is tested to determine one or more pathogens and conditions of the blood. One or more catalysts are automatically added into the blood of the patient. The one or more catalysts are activated utilizing the one or more emitters configured to interact with the one or more catalysts in the blood create treated blood. One or more additives are added into the threated blood. The treated blood is reinjected into the patient.

### BACKGROUND OF THE INVENTION

The present invention relates to blood decontamination. More specifically, but not exclusively, the illustrative embodiments relate to a process for clearing blood of active pathogens.

For many years, blood donations have been treated to perform decontamination and pathogen removal. However, many of these processes require extensive processing and techniques that are not cost effective. For example, blood may be treated utilizing extremely expensive machinery that may require multiple hours. Many of these systems are inflexible, expensive, and require ideal donors, recipients, or other patients in ideal circumstances. Existing systems have struggled to provide timely and effective treatment of blood that may apply to numerous patients.

### SUMMARY

One embodiment of the illustrative embodiments provides a system, device, and autologous method for treating blood. Blood is extracted from a patient. The blood is tested to determine one or more pathogens and conditions of the blood. One or more catalysts are automatically added into the blood of the patient. The one or more catalysts are activated utilizing the one or more emitters configured to interact with the one or more catalysts in the blood create treated blood. One or more additives are added into the threated blood. The treated blood is reinjected into the patient.

### BRIEF DESCRIPTION OF THE DRAWINGS

Illustrated embodiments of the present invention are described in detail below with reference to the attached drawing figures, which are incorporated by reference herein, and where:

FIG. 1 is a pictorial representation of a system for managing blood data in accordance with an illustrative embodiment;

FIG. 2 is a pictorial representation of a blood processing system in accordance with an illustrative embodiment;

FIG. 3 is a cross-sectional view of tubing utilized in the treatment module in accordance with illustrative embodiments;

FIG. 4 is a side view of the tubing of FIG. 3 in accordance with an illustrative embodiment;

FIG. 5 is an intravenous (IV) bag equipped with a light source in accordance with an illustrative embodiment;

FIG. 6 is a flowchart of a process for treating blood in accordance with an illustrative embodiment;

FIG. 7 is a flowchart of a process for treating blood in accordance with an illustrative embodiment;

FIG. 8 is a flowchart of a process for compiling data and scores associated with treated blood in accordance with an illustrative embodiment; and

FIG. 9 depicts a computing system in accordance with an illustrative embodiment.

### DETAILED DESCRIPTION OF THE PREFERRED EMBODIMENTS

The illustrative embodiments provide a system, method, and device for a donor-based blood decontamination. The decontamination is performed to remove pathogens using a range of catalysts and activators using different techniques from a single patient, donor, cadaver, or other sources elicit, measure, and improve an immunity response for a patient. The illustrative embodiments may utilize artificial intelligence, machine learning, algorithms, and other processes to select and utilize catalysts and activators.

The illustrative embodiments are agnostic in nature, which provides for cross-utilization and cross-comparison of catalysts and activators. This is accomplished due to the fact that these processes provide a similar or generalized efficiency and effectiveness in the blood decontamination and pathogen removal process. The illustrative embodiments provide an improved process to test, measure and score best practices and subtle technique refinements for improved pathogen reduction through a data documentation and refinement process that informs the data, information, and logic utilized by artificial intelligence.

A cloud-based system and server may be used to measure and score effectiveness across the varied techniques, utilizations and specialized processes applied to the range of catalysts and activators selected for pathogen reduction. The illustrative embodiments provide the ability to measure and prove out best practices across a wide range of pathogen reduction techniques, catalysts, activators and processes, which may be weighted and compared to create an improved process utilizing optimized data driven components, which are used to measure and score the processes, techniques and system efficiency of pathogen reduction in blood samples.

The illustrative embodiments provide a data and process documentation system which is used to document a variety of factors related to the inclusion or exclusion of a single activators and/or catalysts or various combinations of activators and/or catalysts, utilizations, combinations, types, reaction rates, reaction times, reaction measurements, ultraviolet (UV) light pulse rates, UV intensity, and so forth.

In one embodiment, the utilization is used to document the process efficacy across a variety of known pathogen reduction techniques using UV light sources as the activator in combination with a range of known catalysts. Thus, creating the ability to apply a cross comparison of past and current process utilization and efficacy data providing an effective process to inform, measure and weight the process refinement recommendations across a wide range of activator and catalyst types.

Data analysis and data capture are used to provide measurable efficacy providing the ability to discover and document new best practices in the decontamination of blood, and virus and pathogen reduction thereby providing an improved low tech, safe, non-toxic, self-testing, self-vaccination, and self-inoculation method. The illustrative embodiments utilize a number of activators used in combination with known catalysts as a method for eliciting and documenting a variety of immunity responses to combat SARS-CoV-2 (COVID-19) with additional applicability across a wide variety of viruses and pathogens. Illustrated in this patent are concepts that specifically address the elicitation of an immunity response in a patient suffering with COVID-19. The processes, catalysts and activators described herein may be interchanged and measured for process effectiveness and are not limited to a specific strain of virus or pathogen. The illustrative embodiments may also be utilized to illicit an immunity response and potentially indicate new immunity responses across a wide range of applicable viruses, pathogens, and diseases.

In the illustrative embodiments, donated blood (e.g., donor, transplant, blood bank, cadaver, etc.) is processed through a pathogen reduction system and method. In one embodiment, riboflavin is photosynthesized utilizing UV light to eliminate pathogens or infected blood. Blood for transfusions may be inactivated by adding riboflavin and irradiating the blood with UV light. As a result, viruses and/or pathogens are inactivated. Inoculated viruses may be reintroduced through an injection, spray, or other exposure to provide an immunity response. The illustrative embodiments including the described devices, systems, equipment, and processes may capture data points, such as active virus(es), inactivated virus(es), antibody response, blood sample, chromosomes structures, biome data, future markers, preexisting conditions, genetic makeups, pathogen markers, genomic outlier identification, diet recommendations, human condition diagnostics, allergies, and so forth.

Data may also be captured regarding pathogens, such as viruses including origination, modifications, variants, propagation methods, comorbidities, and so forth.

The blood type, DNA, blood characteristics, and any number of other information may be utilized to utilize treated blood in a process that is best known to help a patient. For example, family group information, demographic data, and blood type treatment may be compiled over time to provide the most effective treatments possible.

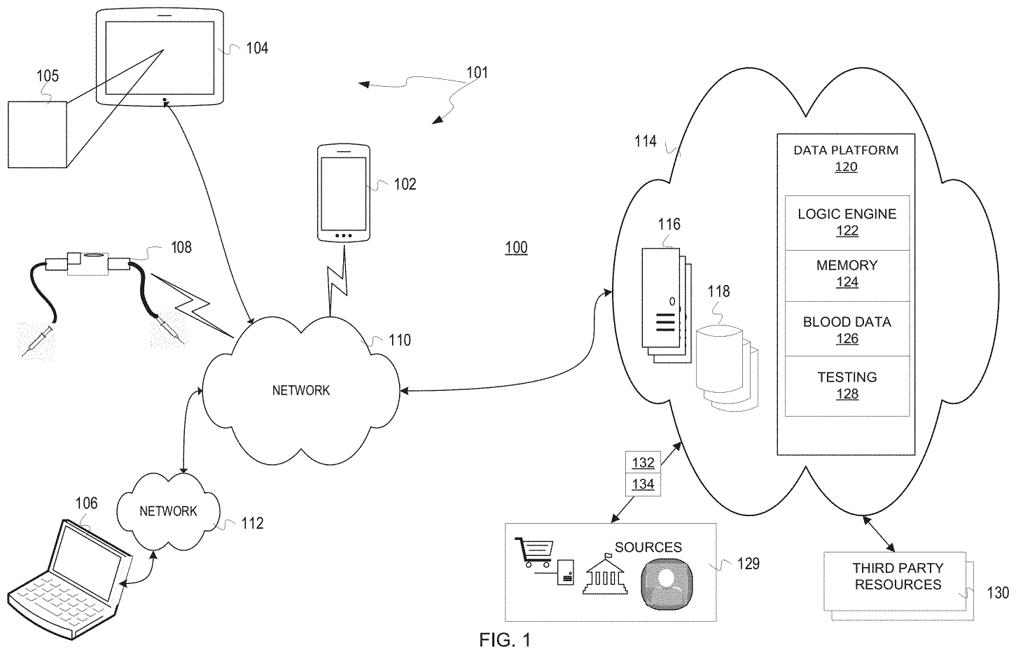

FIG. 1 is a pictorial representation of a system 100 for managing blood data in accordance with an illustrative embodiment. In one embodiment, the system 100 of FIG. 1 may include any number of devices 101, networks, components, software, hardware, and so forth. In one example, the system 100 may include a smart phone 102, a tablet 104 displaying graphical user interface 105, a laptop 106 (altogether devices 101), a blood system 108, a network 110, a network 112, a cloud system 114, servers 116, databases 118, a data platform 120 including at least a logic engine 122, a memory 124, blood data 126, and testing 128. The cloud system 114 may further communicate with sources 129 and third-party resources 130.

Each of the devices, systems, and equipment of the system 100 may include any number of computing, telecommunications, and medical components, devices or elements which may include needles/intravenous interfaces, blood samplers, infusers, centrifuges, processors, memories, caches, busses, motherboards, chips, traces, wires, pins, circuits, ports, interfaces, cards, converters, adapters, connections, transceivers, displays, antennas, operating systems, kernels, modules, scripts, firmware, sets of instructions, and other similar components and software that are not described herein for purposes of simplicity.

In one embodiment, the system 100 may be utilized by any number of users, healthcare providers, organizations, or providers to aggregate, manage, review, analyze, process, and/or tests blood data 126 (e.g., persona/consumer, commercial, etc.). For example, the blood data 126 may be utilized to treat multiple patients. In one embodiment, the patients represent any number of individual humans, pets, mammals, or so forth that may need treatment. The system 100 may represent, connect with, or integrated with one or more hospitals, clinics, nursing homes, research facilities, universities/colleges, treatment facilities, or medical systems. In one embodiment, the system 100 may utilize any number of secure identifiers (e.g., passwords, pin numbers, certificates, etc.), secure channels, connections, or links, virtual private networks, biometrics, or so forth to upload, manage, and secure the blood data 126, testing 128, and perform applicable testing. In one embodiment, the system 100 may be a blockchain system that utilizes a digital ledger to track testing involving the blood data 126, associated diagnostics, or utilization thereof. For example, the digital ledger may store the transaction details, information, and data. The devices 101 are representative of multiple devices that may be utilized by businesses or commercial and consumers. The devices 101 utilize any number of applications, browsers, gateways, bridges, or interfaces to communicate with the cloud system 114, platform 120, and/or associated components.

The blood data 126 may include a number of different data types. The blood data 126 may include blood type, pathogens (e.g., viruses, bacteria, etc.) contaminants, demographic data, patient conditions, family and health data, and other applicable types of data. Demographic data may be a combination of data points that include age, gender, exercise profile, occupation, marital status, education/education level, income level, religion, birthday, family size, and so forth.

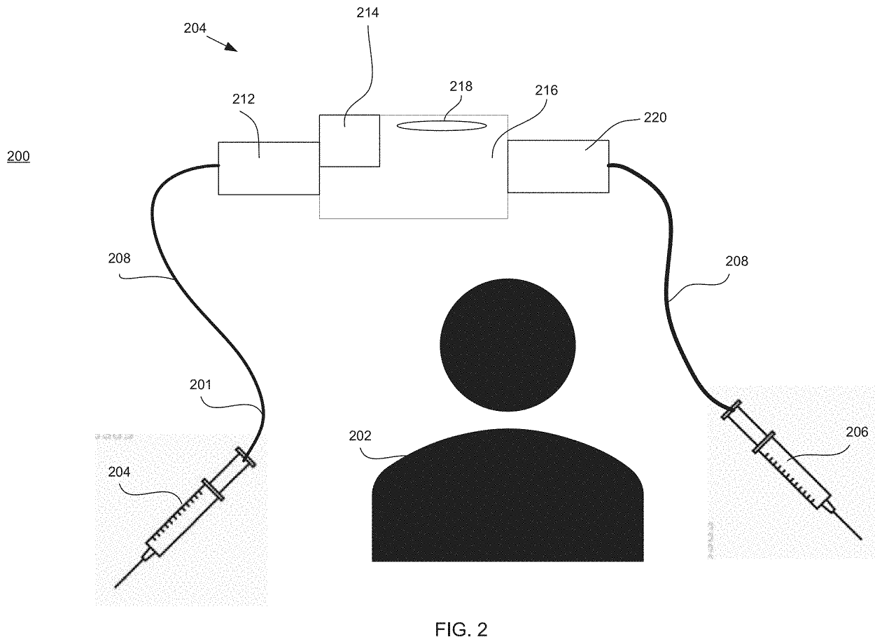

The wireless device 102, tablet 104, and laptop 106 are examples of common devices that may be utilized to receive and manage blood data 126 and perform testing related thereto. In one embodiment, the wireless devices 101 may include a blood sample peripheral (e.g., wireless or wired) that allows the wireless devices 101 to capture measurements. The measurements may be utilized by the wireless devices 1010 or the cloud system 114 to determine pathogens, contaminants, and potential treatments. The wireless devices 101 may utilize any number of existing blood analysis devices or the blood system 108 (see also FIG. 2). Other examples of devices 101 may include e-readers, cameras, video cameras, audio systems, gaming devices, vehicle systems, kiosks, point of sale systems, televisions, smart displays, monitors, entertainment devices, medical devices, virtual reality/augmented reality systems, or so forth. The devices 101 may communicate wirelessly or through any number of fixed/hardwired connections, networks, signals, protocols, formats, or so forth. In one embodiment, the smart phone 102 is a cell phone that communicates with the network 110 through a 5G connection. The laptop 106 may communicate with the network 112 through an Ethernet, Wi-Fi connection, or other wired or wireless connection.

The blood data 126 may be collected and sourced from any number of online and real-world sources including, but not limited to, permitted healthcare data, website traffic and cookie-based analytics, social media, user profiles, application data, location data, government databases, surveys and questionnaires, and other applicable sources. The blood data 126 may include associated treatments, processes, catalysts, and other information relevant to one or more patients, donors, or other applicable parties.

The cloud system 114 may aggregate, manage, analyze, and process blood data 126 across the Internet and any number of networks, sources 129, and third-party resources 130. For example, the networks 110, 112, 114 may represent any number of public, private, government, virtual, specialty, or other network types or configurations. The different components of the system 100, including the devices 101 may be configured to communicate using wireless communications, such as Bluetooth, Wi-Fi, or so forth. Alternatively, the devices 101 may communicate utilizing satellite connections, Wi-Fi, 3G, 4G, 5G, LTE, personal communications systems, DMA wireless networks, and/or hardwired connections, such as fiber optics, T1, cable, DSL, high speed trunks, powerline communications, and telephone lines. Any number of communications architectures including client-server, network rings, peer-to-peer, n-tier, application server, mesh networks, fog networks, or other distributed or network system architectures may be utilized. The networks, 110, 112, 114 of the system 100 may represent a single communication service provider or multiple communications services providers.

The sources 129 may represent any number of web servers, distribution services (e.g., text, email, video, etc.), media servers, platforms, distribution devices, or so forth. In one embodiment, the sources 129 may represent the healthcare providers that purchase, license, or utilize the blood data 126, such as hospitals, clinics, research facilities, and other facilities, groups, and services utilizing the system 100. In one embodiment, the cloud system 114 (or alternatively the cloud network) including the data platform 120 is specially configured to perform the illustrative embodiments.

The cloud system 114 or network represents a cloud computing environment and network utilized to aggregate, process, manage, sell, monetize, and distribute blood data 126 and support the associated testing and utilization. The cloud system 114 allows blood data 126, testing 128, and other applicable information from multiple healthcare providers, doctors, businesses, users, patients managers, or service providers to be centralized. In addition, the cloud system 114 may remotely manage configuration, software, and computation resources for the devices of the system 100, such as devices 101. The cloud system 114 may prevent unauthorized or inappropriate access to blood data 126, testing 128, tools, and resources stored in the servers 116, databases 118, and any number of associated secured connections, virtual resources, modules, applications, components, devices, or so forth. In addition, a user may more quickly upload, aggregate, process, manage, and distribute blood data 126 (e.g., catalysts, additives, treatments, profiles, updates, surveys, content, etc.) where authorized, utilizing the cloud resources of the cloud system 114 and data platform 120. The cloud system 114 allows the overall system 100 to be scalable for quickly adding and removing users, businesses, authorized treatment providers, analysis modules, distributors, valuation logic, algorithms, moderators, programs, scripts, filters, transaction processes, distribution partners, or other users, devices, processes, or resources. Communications with the cloud system 114 may utilize encryption, secured tokens, secure tunnels, handshakes, secure identifiers (e.g., passwords, pins, keys, scripts, biometrics, etc.), firewalls, digital ledgers, specialized software modules, or other data security systems and methodologies as are known in the art. The data platform 120 is used as a vault for healthcare data, personal data, user profile, corporate data and data pools through the use of VPN's, secure networks, firewalls and internet data encryption methodologies that ensure the vaulted data cannot be accessed without user profile permission. Other intelligent network devices may also be utilized within the cloud system 114.

The servers 116 and databases 118 may represent a portion of the data platform 120. In one embodiment, the servers 116 may include a web server 117 utilized to provide a website, mobile applications, and user interface (e.g., user interface 107) for interfacing with numerous users. Information received by the web server 117 may be managed by the data platform 120 managing the servers 116 and associated databases 118. For example, the web server 117 may communicate with the database 118 to respond to read and write requests. For example, the servers 116 may include one or more servers dedicated to implementing and recording testing 128 and communications involving the blood data 126. For example, the databases 118 may store a digital ledger for updating information relating to the user's blood data 126 as well as utilization of that blood data 126 to generate customized treatments. The databases 118 may utilize any number of database architectures and database management systems (DBMS) as are known in the art. The databases 118 may store the content associated with each user which may specify an address, name, age, demographics, diseases/viruses/conditions, interests, family/friend information, biometric identifiers, payment information, permissions, settings, location, and so forth. Any number of secure identifiers, such as tones, QR codes, serial numbers, or so forth may be utilized to ensure that content, personal, or transaction information is not improperly shared or accessed.

The user interface 105 may be made available through the various devices 101 of the system 100. In one embodiment, the user interface 105 represents a graphical user interface, audio interface, or other interface that may be utilized to manage data and information. For example, the user may enter or update associated data utilizing the user interface 105 (e.g., browser or application on a mobile device). The user interface 105 may be presented based on execution of one or more applications, browsers, kernels, modules, scripts, operating systems, or specialized software that is executed by one of the respective devices 101. The user interface may display current and historical data as well as trends. The user interface 105 may be utilized to set the user preferences, parameters, and configurations of the devices 101 as well as upload and manage the data, content, and implementation preferences sent to the cloud system 114.

In one embodiment, the system 100 or the cloud system 114 may also include the data platform 120 which is one or more devices utilized to enable, initiate, generate, aggregate, analyze, process, and manage blood data 126, testing 128, and so forth with one or more communications or computing devices. The data platform 120 may include one or more devices networked to manage the cloud network and system 114. For example, the data platform 120 may include any number of servers, routers, switches, or advanced intelligent network devices. For example, the data platform 120 may represent one or more web servers that performs the processes and methods herein described.

In one embodiment, the logic engine 122 is the logic that controls various algorithms, programs, hardware, and software that interact to receive, aggregate, analyze, rank, process, score, communicate, and distribute blood data 126, content, testing 128, alerts, reports, messages, or so forth. The logic engine 122 may utilize any number of thresholds, parameters, criteria, algorithms, instructions, or feedback to interact with users and interested parties and to perform other automated processes. The logic engine 122 may represent a processor. The processor is circuitry or logic enabled to control execution of a program, application, operating system, macro, kernel, or other set of instructions. The processor may be one or more microprocessors, digital signal processors, application-specific integrated circuits (ASIC), central processing units, or other devices suitable for controlling an electronic device including one or more hardware and software elements, executing software, instructions, programs, and applications, converting and processing signals and information, and performing other related tasks. The processor may be a single chip or integrated with other computing or communications elements.

The memory 124 is a hardware element, device, or recording media configured to store data for subsequent retrieval or access at a later time. The memory 124 may be static or dynamic memory. The memory 124 may include a hard disk, random access memory, cache, removable media drive, mass storage, or configuration suitable as storage for blood data 126, testing 128, treatments, instructions, and information. In one embodiment, the memory 124 and logic engine 122 may be integrated. The memory 124 may use any type of volatile or non-volatile storage techniques and mediums.

In one embodiment, the cloud system 114 or the data platform 120 may coordinate the methods and processes described herein as well as software synchronization, communication, and processes. The third-party resources 130 may represent any number of human, automatic, or electronic resources utilized by the cloud system 114 including, but not limited to, healthcare providers, government groups, businesses, entities, organizations, individuals, government databases, private databases, web servers, research services, and so forth. For example, the third-party resources 130 may represent the

Centers for Disease Control, healthcare aggregators, Doctor's groups, treatment coordinators, health insurance providers, and others that contribute to, manage, aggregated, and/or pay for rights to use the blood data 126.

In one embodiment, the data platform 120 may implement a blockchain ledger, manager, or technology. In another embodiment, the blockchain ledger may be accessible through sources 129. Any number of existing blockchain companies or providers may be utilized (Aeternity, Ethereum, Bitcoin, Dfinity, ContentKid, Blockphase, Chain of Things, Flowchain, Decissio, Cognate, SkyHive, Safe, etc.).

The blockchain is utilized as a way to store and communicate the blood data 126 along with testing 128. The blockchain may utilized one or more distinct ledgers for different entities, services providers, types of data, users, or so forth. For example, each new user with data received by the data platform 120 is assigned a token or other secure identifier. In one embodiment, the digital tokens may be managed utilizing a key that allows the user or controlling party to access the ledger. In one example, the tokens may be controlled by the user or control may be reassigned. The blockchain may cross-reference updates to the blood data 126 with the original record for the data platform 120 to ensure proper maintenance, control, licensing, management, and testing. In one example, different licensing tiers, pricing algorithms, license verification, cause information, and payments are combined to create a unique platform. The illustrative embodiments provide a system 100, cloud system 114, and data platform 120 for compiling businesses that support causes and documenting commercial and consumer testing that support those causes.

The third-party resources 130 may represent any number of electronic or other resources that may be accessed to perform the processes herein described. For example, the third-party resources 130 may represent government, private, and charitable servers, databases, websites, services, and so forth for generating, aggregating, and managing the blood data 126.

The logic engine 122 may perform analysis of the blood data 126. For example, all the of the global resources and information, such as effective treatments by virus, blood type, condition, demographics, location, and other information, factors, criteria, and characteristics may be shared. The logic engine 122 may also track communications, access, sales, or transfers of blood data 126 between one or more groups. The logic engine 122 may establish and map cross catalyst and activator testing based on various combinations, treatment processes, and other information as part of the blood data 126. As a result, treatment maps may be generated and shared for numerous patients, donors, and users. The blood data 126 and testing 128 may be utilized to provide improvements around data utilization and mapping for the purpose of blood type matching, source matching, harvesting capability, immunity response identification, contract tracing, and disease spread. The system 100 may also include refrigeration or cryogenic units that manage blood storage, access, testing, treatment and so forth. As a result, the system 100 may be used in storing, mapping, and tracking blood (e.g., temporary, or cryogenic blood storage). The blood may be tested, treated, stored, mapped, and tracked for any number of patients utilizing blood type, family members, blood characteristics, DNA, and/or other factors. The system 100 may facilitate the matching of transfusion recipients who require a specific donor type or treated blood. For example, specific antigens or antibodies may be required for an immunity response.

The logic engine 122 may process permitted data feeds and information received to capture blood data 126. For example, the logic engine 122 may access sources 128 including healthcare exchanges, peer-approved research, scientific journals, markets, consultants, management systems, and so forth. For example, current and historical values for blood data 126 may be determined and utilized in real-time.

The illustrative embodiments may also support third-party analysis of the blood data 126. The validations may be performed by auditing groups, commissions, industry groups, or other professionals/entities. In one embodiment, the sources 129 may determine or verify blood data 126 analysis and testing 128. Any number of artificial intelligence or computerized processes may be utilized to generate, test, and validate the blood data 126. The sources 129 may also aggregate blood data 126 into portfolios. Portfolios of data may be managed, transferred, and otherwise utilized for the benefit of the data owners.

In one embodiment, the logic engine 122 may utilize artificial intelligence. The artificial intelligence may be utilized to enhance blood data 126 and increase its effectiveness. For example, artificial intelligence may be utilized to review, authenticate, and validate blood data 126 that is received by the system 100. The artificial intelligence of the logic engine 122 may be utilized to ensure that the blood data 126 is improved, accurately analyzed, and efficiency increased.

In another embodiment, the devices 101 may include any number of sensors, appliances, and devices that utilize real time measurements and data collection to update the blood data 126. For example, a sensor network, wearables (e.g., watches, bands, implantable devices, etc.) and Internet of things (IOT) devices may gather user and behavioral data.

The data platform 120 may capture known data, behavioral information, psychological, mood data, and other intangible data. The blood data 126 may be validated through artificial intelligence, machine learning, human analysts/consultants, or other automated or manual processes. For example, the system may be utilized to document participation and track results and side effects from a medical trial. The effective use of the blood data 126 may be rated for individuals, companies, facilities, or others. Data waste and data proficiencies may be managed through the data platform 120.

FIG. 2 is a pictorial representation of a blood processing system 200 in accordance with an illustrative embodiment. In one embodiment, the blood processing system 200 may represent the blood system 108 of FIG. 1. The blood processing system 200 may be utilized to treat blood 201 of a patient 202. The blood processing system 200 may be a closed system. The blood processing system 200 may include needles 204, 206, tubing 208, a blood platform 210 including testing module 212, additive module 214, treatment module 216, light source 218, and additive module 220. In one embodiment, the blood processing system 200 may be controlled by systems, devices, or platforms, such as the cloud system 114, data platform 120, or wireless devices 101 of FIG. 1.

The various components and modules of the blood processing system 200 may be combined in any order. Additional modules may also be added to the blood processing system 200. Modules, components, and other aspects of the blood processing system 200 may also be removed. The patient 202 may represent a single patient being treated or a volunteer whose blood is being tested. In some embodiments, blood 201 removed from the patient 202 is not returned to the body of the patient 202 as the blood 201 is treated for experimental, research, or testing purposes only. The patient 202 may alternatively represent multiple patients, a donor, and a receiver, or so forth. The blood 201 may represent blood received from a living or recently deceased patient. The blood 201 may also represent any number of blood analogues, blood simulants, fluids, plasma, medicines, or so forth.

In one embodiment, the needles 204, 206 and tubing 208 represent a blood transfer system. For example, the blood 201 of the user 202 may be moved from one arm to another after being treated by the blood processing system 200. In one embodiment, testing module 212 may be utilized to test the blood 201 of the user 202 for pathogens, viruses, contaminants, antibodies, and so forth. The testing module 212 may also measure blood characteristics (e.g., composition, structure, status, levels, percentage, volume, flow, condition, etc.), such as temperature, viscosity, platelet/thrombocytes counts, color, type, oxygen levels, nutrients (e.g., iron), blood sugar/glucose, red blood cell/erythrocytes count, white blood cells/leukocytes, protein levels, and other applicable information. The testing module 212 may also capture the DNA, antibody response, and other applicable information. The testing module 212 may track DNA information for a number of patients, family members, and so forth to track responses to treated blood as herein described. As a result, past results may be applied to future needs throughout the lifecycle of individuals, families, demographics, and groups. The testing module 212 may also be operated independently to sample blood, tissues, stem cells, cord blood, fluids, or other samples.

In one embodiment, the needles 204, 206 may be formed of transparent, semi-transparent, translucent, or semi-translucent materials, such as glass, plastic, quartz, polyplastics (cyclic olefin copolymer). The hollow needles 204, 206 may communicate blood or fluids through their center while acting as a light guide or communications medium for applying ultraviolet light (e.g., UV-A, UV-B, UV-C, etc.). As a result, any number of optical, infrared, or ultraviolet emissions may be applied through the needles 204, 206 for interaction with the blood, catalysts, additives, or so forth. The light source may be integrated or attached to the needles 204, 206 or may be connected through a light guide, such as a fiber optic, channel, or so forth. In another embodiment, the needles may connect to a syringe or expanded portion integrated with the needles 204, 206. The syringe or expanded portion may have a light source or emitter integrated for applying one or more emission types. Emitters include, for example, a light source including, for example, UV-A, UV-B, or UV-C light.

The additive module 214 may be utilized to add any number of catalysts (e.g., vitamins, minerals, medications, nanoparticles, stimulants, or other materials to the blood 201. In another embodiment, infectants (e.g., inactive pathogens, feces, etc.) may be added to initiate a pathogen reduction process. The additive module 214 may also mix the blood 214 with the catalysts. Mechanical mixing, injections, agitation, or other processes may be utilized in any number of receptacles, mediums, or containers to mix the blood with the catalysts. In another example, an interactive agent, such as ozone, may be applied to elicit a immunity response or as an inactivator.

The treatment module 216 may be utilized to treat the blood 201. The light source 218 may represent any number of lights, bulbs, heaters, wave, or signal generators (e.g., optics, electromagnetic signals, radio frequencies, sounds, ultrasonic waves, infrared waves, etc.), X-rays, or so forth. For example, the light source 218 may represent UV-A, UV-B, UV-C, or other spectra of UV light. The light source 218 may also emit gamma radiation, laser light, sound magnetic fields, charged particles/free energy, free radicals, mechanical vibrations, or other emissions. The emissions may be performed consistently, in pulses, intermittently, randomly, in a pattern, or so forth. The spectra, wavelengths, frequency, amplitudes, sequences, rates, waves, and harmonics may be adjusted to maximize the catalyst/additive interactions with the activator. Any number of resonant frequencies or harmonics may be utilized. The treatment module 216 may include shielded walls or separators for protecting the patient 202 from exposure to the emissions from the light source 218. The treatment module 216 may also or alternatively include a vibrator or vibration component (mechanical, sonic, RF, etc.) for activating the catalyst, mixing/agitating the blood, and performing treatments.

The additive module 220 may add any number of additives or activators to the blood 201. For example, any number of the 13 essential vitamin groups (i.e., A, C, D, E, K and the B vitamins thiamine (B1), riboflavin (B2), niacin (B3), pantothenic acid (B5), pyridoxine (B6), biotin (B7), folate (B9) and cobalamin (B12)) and subgroups may be added, such as A2 retinol, retinyl esters, retinal, and/or combinations of known activators, vitamins, and minerals. The additive module 220 may add any number of activators or catalysts that interact with the treatment module 216 and/or light source 218. Additionally, the blood processing system 200 may utilize single non-vitamins or catalyst combinations which are capable of eliciting an immunity response or from naturally occurring non-vitamin sources such as O₃, DMT, Cobalt 60, and other compounds known to decontaminate transfusion blood. The additive module 220 may permanently or temporarily add formaldehyde, ozone, or other compounds to the blood 201 of the patient 202. A varied combination of additives and activation sources (i.e., light source 218) may be utilized, measured, and scored across a range of diseases, conditions, and factors to create a treatment map and efficacy ranges across each tested catalyst, activation source, and disease. In one embodiment, the additive module 220 may utilize heparin or other anti-coagulants which expedite and eliminate the need to freeze the blood.

The additive module 220 may also perform additional testing. For example, the additive module 220 may include all or portions of the testing equipment, devices, and components of the testing module 212. For example, the additive module may ensure that the blood temperature, viscosity, and other factors are acceptable.

The value of utilizing endogenous or naturally occurring compounds is advantageous due to the fact that they are difficult to overdose or overuse and are generally considered safe for human ingestion or infusion. This is due to the fact that they are naturally found in the human or mammalian body. The various additives may be easily absorbed, synthesized, or otherwise processed in various portions of the body.

The illustrative embodiments may establish and map cross catalyst and activator testing of a single source of combinations of catalysts, activators, and additives to provide best practices for pathogen reduction through irradiation using various catalysts and light sources (e.g., UV, radio frequency, infrared, etc.).

The illustrative embodiments allow cadaver blood to be preserved and later utilized to increase blood supplies in a nation or worldwide enabling a greater capacity and volume of viral loads available for inoculation. The illustrative embodiments allow for the activation of an immunity response from cadaver blood that contains an active virus or pathogen through the catalyst and activator interaction and conversion of that blood into an inactivated viral inoculation. This is a key improvement for potentially creating a larger supply of antigen doses from a single donation of cadaver blood. With one cadaver blood donation of 5,000 ml of infected blood utilizing the catalyst and activation process, the illustrative embodiments may potentially harvest one thousand (1,000) vaccinations from a single cadaver blood donation. In one example, four hundred (400) organ donors may provide 200,000 to 400,000 inoculations coming from a virus hotspot. Cadaver blood is usable for 6-8 hours with some reports showing blood usability 24 hours after the patient is deceased. Additionally, the process may be utilized to preserve blood, organs and tissue potentially increasing the time frame for usability.

The illustrative embodiments transforms the blood donation industry by uniting blood suppliers, united blood services, Red Cross, undertakers, phlebotomist, and other similar jobs and transforming these industries and skillsets into potential vaccinology and immunology careers and services.

Postmortem inter-circulatory system infusion with riboflavin or other known catalyst mixing provides a novel and key improvement in the embalming process. For example, saline, riboflavin, or other catalysts may be utilized. This is an improve process on the traditional utilization of formaldehyde as the embalming solution. The postmortem saline and riboflavin and/or catalyst solution provides an improvement that allows for the solution to premix in the circular system as it pushes the solution through the circulatory system during embalming, further preserving, blood, tissue, and organs. In the illustrative embodiments, the blood does not need centrifuging and never interacts with oxygen keeping the blood in a whole blood state, which keeps the blood more sterile and contaminant free.

An additional improvement allows blood banks to better regulate the sterilization process for donor blood, increase donors, and improve blood transfusion safety measures. The current blood bank process does not sterilize blood and only checks for a specific number of identified diseases and offers the available donated blood on a first come first serve basis. The illustrative embodiments may change the way blood is viewed and processed for donation postmortem expanding the world blood shortage, by invoking an outlook that considers that every ounce of blood is valuable and is essential for life.

A future consideration is that over time the current process of body, organ, and blood disposal may be viewed as wasteful or less desirable to dispose of blood in traditional and known compliant blood disposal methodologies such as a hazardous waste, or sinks and drains. The illustrative embodiments may be utilized to share postmortem blood to promote life via organ and blood donation. As a result, blood donations may be utilized to treat patients in near real-time (e.g. within a few hours or days) providing benefits to any number Future considerations include the potential that we could see a legal requirement or moral obligation that postmortem blood and organs should be donated.

One embodiment utilizes a documentation, accounting and contract mechanism that provides the ability to pre-donate and establish a contract for pre or post death monetization of organs, tissue, and blood donations as a means to increase organ and blood donations worldwide.

In another embodiment, the blood donations may be used to inoculate and then cryofreeze a loved one's blood for cloning, DNA, whole blood processes, and protection against future pandemics. The donors may pre-determine a contract that may assist in the monetization of each pint of blood donated postmortem. Alternatively, the donor may select that their family be paid postmortem providing a mechanism where the donor is compensated postmortem with the total payout based on the total number of vaccines created. The donor or the donor family may be compensated based on the final utilization of donated blood, tissue, and organs. The illustrative embodiments provide added advantages for blood preservation, utilization, and maximization allowing for donations to be more effectively decided and performed while living or once deceased.

The patent provides added advantages and novel improvement for blood preservation and increasing blood donation availability by allowing for the ability for a patient to decide to donate blood postmortem, or while they are living.

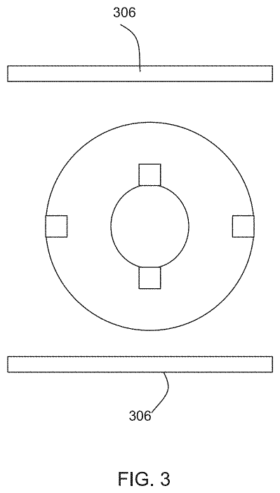

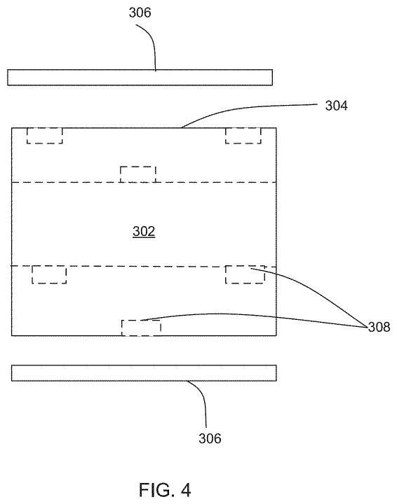

FIG. 3 is a cross-sectional view of tubing 300 utilized in the treatment module in accordance with illustrative embodiments. FIG. 4 is a side view of the tubing of FIG. 3. With regard to FIGS. 3 and 4, the tubing 300 carries blood into a system/device for treatment (not shown). The tubing 300 may be an integral process of the blood treatment. The tubing 300 may be defined around a light source 302. The light source 302 may represent an ultraviolet string light, bulb, ultraviolet light emitting diodes, or other light, wave, heat, radiation, radio frequency, or emission source. The tubing 300 may be positioned within a treatment module. An exterior surface 304 of the tubing 300 may be exposed to one or more light sources 306, frequencies, or emissions. The tubing 300 may be transparent or translucent allowing the blood within the tubing 300 to be exposed to the applicable light source 302 or emissions from an interior portion of the tubing 300 and the exterior surface 304.

In one embodiment, an interior portion of the tubing 300 may include diverters 308. The diverters 308 cause turbulence and flux within the tubing 300 so that additives are mixed, and more portions of the blood are exposed to the light source 302 and the light sources 306. As a result, the blood may be more quickly and more effectively exposed to ultraviolet light or other emissions. The diverters 308 may represent protuberances, channels, ridges, tabs, or other small structures within the tubing 300 that mix the blood and expose more portions of the blood to the light sources 302, 306. The diverters 308 may also be transparent so that increased portions of the blood may be exposed to ultraviolet light.

In one embodiment, the tubing 300 may be positioned in a coil shape or may take a serpentine path. The tubing 300 may be positioned to maximize the exposure to the light source 302, 306.

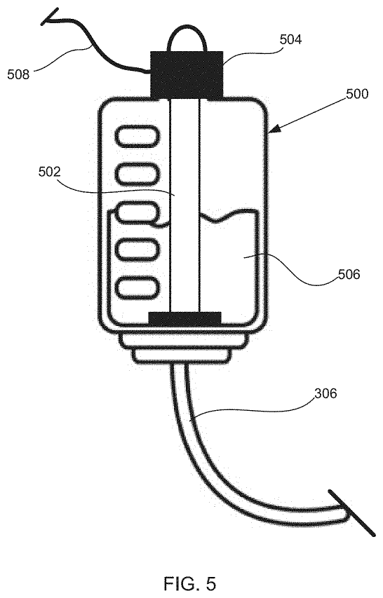

FIG. 5 is an intravenous (IV) bag 500 equipped with a light source 502 in accordance with an illustrative embodiment. In one embodiment, the intravenous bag 500 includes a cap 504 at the top of the IV bag 500 for adding and removing the light source 502. The cap 504 may include a power source 508 including electronics for powering the light source 502 (e.g., ballasts, transformers, fuses, etc.). For example, the light source 502 may plug into a wall outlet utilizing the power source 508 and ballasts within the cap 504 may power and adjust electricity to the light source 502. In another embodiment, a battery may power the light source 502 of the UV bag 500. In another embodiment, the light source 502 may also connect to a power source at a bottom portion of the IV bag 500 to power the light source 502. The light source 502 may treat fluids 506 within the IV bag 500. For example, the fluids 506 may represent blood from a patient that is being treated before being infused back into the patient. Alternatively, the fluids 506 may be from a donor and being communicated to a patient/recipient.

In one embodiment, the cap 504 may also include a motor for moving the light source 502 within the fluids 506. The motor may agitate the fluids 506 to provide better exposure to the light or emissions generated by the light source 502. The motor may stir, vibrate, or otherwise rotate the light source 502. As previously disclosed, the light source 502 may generate ultraviolet light, visible light, ultrasonic waves, radio frequency waves, magnetic fields, electromagnetic waves/fields, radiation, heat, vibrations, sound waves, or so forth. In one embodiment, the light source 502 acts as an activator for one or more catalysts in the fluids 506.

The IV bag 500 may connect to any number of incoming and outgoing tubes, ports, or interfaces, such as the tubing 510. In one embodiment, one or more pumps (not shown) may be utilized to fill the UV bag 500. As a result, the fluids 506 may be added to the UV bag 500 and treated. In another embodiment, the cap 504 or a different port may be utilized to add catalysts or additives to the fluids 506. In one embodiment, the catalysts or additives may interact with one or more of the fluids 506, light source 502, or other portion of the UV bag 500 to prepare the fluids 506 for utilization.

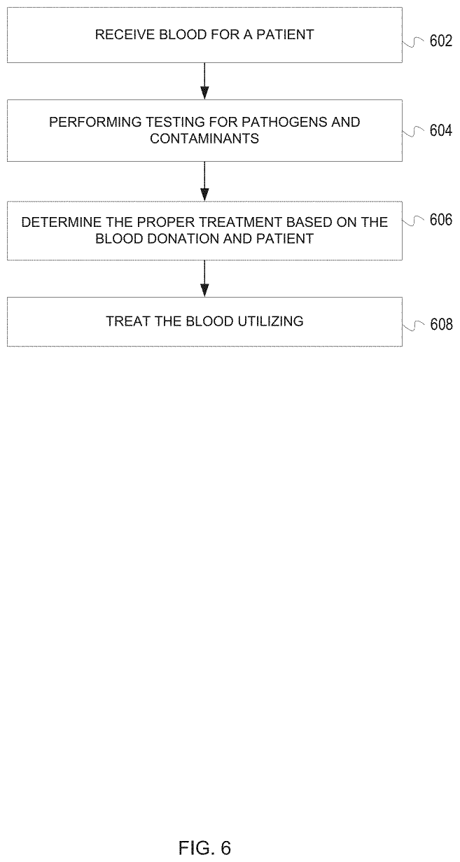

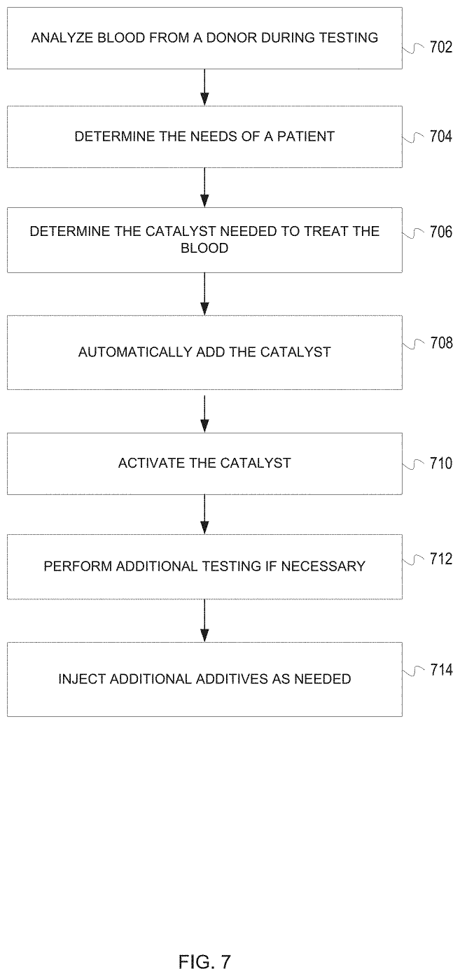

FIG. 6 is a flowchart of a process for treating blood in accordance with an illustrative embodiment. The process of FIGS. 6 and 7 may be implemented by a system (e.g., system 100, blood system 108, 200), such as those described in FIGS. 1-3. All or portions of the process of FIGS. 6-8 may be performed automatically by the system. For example, testing and determinations of applicable catalysts, activators, and additives (e.g., exposure, time periods, amounts, mixing, intensity/amplitude, frequency, etc.) may be automatically performed by the logic of the system. In one embodiment, an autologous system may extract, test, process, and reinject the blood as described in the illustrative embodiments within five minutes, 15 minutes, 30 minutes, or one hour.

The process may begin by receiving blood for a patient (step 602). The blood may be received from the patient himself/herself, a donor (e.g., family member, friend, anonymous donor, etc.), a cadaver, or other party. The blood may also represent a blood analogue or simulant. The blood may also represent blood byproducts, plasma, or other fluids that combine blood and medications, catalysts, or other additives. The blood may be for a person or animal.

Next, the system performs testing for pathogens and contaminants (step 604). The blood test may be performed automatically in response to a sample, or blood flow received during step 602. In one embodiment, testing must be performed and completed before any other steps may be initiated. Any number of tests may be performed based on the blood, the donor, the recipient patient, known diseases, pathogens, conditions, factors, or so forth (e.g., donor or recipient).

Next, the system determines the proper treatment based on the blood donation and patient (step 606). The proper treatment may include the addition or removal of any number of elements, components, or portions of the blood. For example, one or more catalysts may be added to the blood for activation by the system. For example, riboflavin may be mixed into the blood at desired levels and the blood may be exposed to ultraviolet light to denature, kill, or deactivate any number of pathogens or contaminants

Next, the system treats the blood (step 608). The blood may be treated with any number of additives/catalysts, activation emissions (e.g., ultraviolet light, visible light, heat, radiation, magnetic fields, radio frequencies, vibrations, sound waves, etc.). In addition, any number of vaccines, antibodies, antigens, or other components may be added to the blood after being treated.

In one embodiment, the process of FIG. 6 is performed in real-time for a single patient. For example, blood may be withdrawn from a first arm of the user and then injected back into the second arm of the user. In other embodiments, the blood may be moved from a donor to a recipient. The donor may be living or deceased.

FIG. 7 is a flowchart of a process for treating blood in accordance with an illustrative embodiment. In one embodiment, the process of FIG. 7 may represent steps, such as steps 606 and 608 of FIG. 6. The process may begin by analyzing blood from a donor during testing (step 702). The blood may be analyzed for any number of pathogens, conditions, factors, contaminants, elements, and so forth.

Next, the system determines the needs of a patient (step 704). As previously noted, the donor and the patient/recipient may be the same person. Alternatively, the donor and the patient may represent distinct individuals. The patient may need to have specific vitamins, medicines, antibodies, antigens, red blood cell counts, plasma content, or so forth.

Next, the system determines the catalyst needed to treat the blood (step 706). The system may utilize a matrix or algorithm of applicable information to determine the catalyst. For example, the catalyst may be selected based on blood type (e.g., donor, patient/recipient etc.), factors, diseases/conditions, deficiencies, availability, and so forth. In one specific embodiment, the catalyst is riboflavin. Riboflavin is a B vitamin. Riboflavin is involved in many processes in the body and is necessary for normal cell growth and function. Riboflavin (also known as vitamin B2) is one of the B vitamins, which are all water soluble. The utilization of riboflavin is favored because it naturally absorbs into a virus and binds to Ribonucleic Acids (RNA). Riboflavin also provides unique advantages, such as quick absorption into the body and it is virtually impossible to overdose or cause long term harm due to the rapid rate that the body may flush the Riboflavin out of the patient's system.

Next, the system automatically adds the catalyst (step 708). The catalyst may be added in tubing, a bag, or other receptacle or medium. During step 708 (or separately), the system may mix the catalyst with the blood. Any number of mixing, stirring, flux, or other processes may be utilized. Mixing, integration, or injection of the catalyst into the blood may be performed in tubing, a mixing chamber, IV bags, or other receptacle or communications mediums within the system.

Riboflavin (vitamin B2) is activated by the ultraviolet (UV) light source, produces active oxygen which damages cell membrane and prevents replication of the carrier of diseases (viruses, bacteria, protozoa, leucocytes) in all blood products. The aim of this study was to establish the influence of the process of pathogens photoinactivation using riboflavin and UV rays on the biochemical and functional characteristics of platelet concentrates prepared from “buffy coat”.

Next, the system activates the catalyst in (step 710). The catalyst may be activated utilizing one or more emissions (e.g., optical, infrared, radio frequency, magnetic, x-ray, radiation, sound waves, vibrations, etc.). In one embodiment, the emissions may be ultraviolet light applied to the blood and integrated catalysts. The activators may be applied from any number of sources, directions, or locations to best interact with the blood and catalysts. The activators and associated emissions may also be applied in different intensities, strength, frequencies, spectra, or so forth. In one embodiment, the blood sample may be exposed to ultraviolet light for 2-4 minutes per pint. For example, 60 mL of blood may be mixed with 10 mL of riboflavin for treating a COVID patient. In another embodiment, activation of the catalyst may be using one or more chemical (e.g., liquids, solids, gases etc.). In some embodiments, the catalyst may be highly sensitive to interactions, such as light interactions. The activation process may create a reaction which breaks down pathogens, such as viruses. For example, riboflavin mixed with the blood to a cause a vibration that breaks down the Corona virus (or other viruses) to help treat the patient. In one example, an entire process may be performed for a patient within thirty minutes to an hour.

Next, the system performs additional testing necessary (step 710). The system may test the blood once the catalysts have been added and activated utilizing one or more processors. For example, the system may determine if pathogen levels are eliminated or decreased. The system may also test to determine whether contaminants have been removed, transitioned, or neutralized. The system may also determine whether the mixture or combination of the catalyst is effective as determined during step 706.

Next, system injects additional additives as needed (step 714). The system may add additives that may have been negatively affected by the activation process of step 710. For example, a vaccine, blood treatment, or other materials may be added or mixed with the blood. The vaccine is a substance used to stimulate the production of antibodies and provide an immunity against one or more diseases. The vaccine may be prepared from causative agent of a disease, its products, or a synthetic substitute, treated to act as an antigen without inducing the disease. The vaccine may include nonliving microorganisms, leaving attenuated organisms, or living fully virulent organisms that are administered to produce or artificially increase immunity to a particular disease, issue, or condition. The catalyst and additives thorough testing, analysis, approval (e.g., FDA, governmental, industry, group, etc.) and analysis prior to being utilized in the illustrative embodiments. At the end of the process of FIG. 7, the blood is considered treated blood or processed blood.

The system may also transfuse, inject, or infuse the treated blood treated during the process of FIG. 7 into the patient/recipient. The process blood may be transfused into a patient (e.g., COVID, flu, strep throat, pneumonia, vitamin deficient, etc.) to illicit an inoculated or medicated immunity response.

In another embodiment, the catalysts added in step 708 may be Vitamin A2, B6, C, D, or K. Vitamin K may be used as a catalysts for eliciting an immunity response. Vitamin K may act as a cofactor for some plasma proteins thereby affecting immune and inflammatory responses particularly mediated by T cells. Vitamin K may inhibit cell growth by promoting apoptosis and autophagy. There are indications of strong interaction with vitamin K and infrared light, UV (e.g., UV-A, UV-B, UV-C, etc.) light, gamma radiation, or other activators to illicit an immunity response.

In another embodiment, the catalyst may represent ozone (O₃) or O₄ into the circulatory system through blood to mount an immune response. Ozone may be used to either suppress or enhance an immune response based on the condition, circumstances, blood type, infections/pathogens, and other criteria.

The illustrative embodiments are hands free an autologous. As a result, once one or more needles are inserted into the patient the process may be performed to treat the patient's blood. In other embodiments, the blood may be treated in real-time to provide real-time

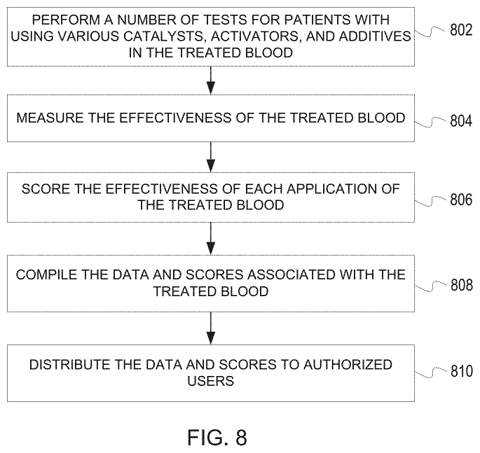

FIG. 8 is a flowchart of a process for compiling data and scores associated with treated blood in accordance with an illustrative embodiment. The process may begin by performing a number of tests for patients using various catalysts, activators, and additives in the treated blood (step 802). The tests may include performing the processes of FIGS. 6 and 7. Tests may be performed utilizing any number of clinical trials (phases 0-4), cohort testing, and other types of testing. The testing may be performed specific to donors, recipients, demographics, pathogens/contaminants/conditions/issues/diseases, delivery method, combinations, amounts, or other factors. The testing may be performed varying the catalysts, activators, and additives to different levels, types, and combinations.

Next, the system measures the effectiveness of the treated blood (step 804). The measurements may be made regarding the status of the pathogens/contaminants in the treated blood, the response of the patient, and any number of other factors. For example, any number of objective criteria utilized by healthcare workers and facilities may be utilized. Measurements of the biological and immunity response of the living cells within the blood and/or patient may be gathered. The measurements may be taken utilizing any number of devices, systems, or equipment.

Next, the system scores the effectiveness of each application of the treated blood (step 806). Each measured outcome from step 804 may have varied results depending on the catalyst and activator source selections and process refinements.

Next, the system compiles the data and scores associated with the treated blood (step 808). The data, scores, and other information associated with the treated blood may be saved. For example, the data may be saved in a cloud system for distribution to any number of parties. The data is compiled so that the blood treatment process may be refined to improve a patient ability recover or improve based on the treatment. For example, the blood treatment may be analyzed to ensure the patient exhibits a slow and long lasting hyper stimulated immunoglobulin response.

Next, the system distributes the data and scores to authorized users (step 810). The data, scores, and other relevant information determined during the process of FIG. 8 may be distributed to users, devices, organizations, facilities, systems, servers, software systems, parties, or other individuals or groups.

The illustrative embodiments provide a measured data driven process that is less labor intensive, less expensive, and providers quicker and more nimble deployment at onsite and mobile destinations. The illustrative embodiments have applications in hospitals, care facilities, intensive care units, convalescence, mortuaries, nursing homes, hospices, aid facilities, non-medical facilities/settings, and other locations, facilities, and organizations.

The illustrative embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, embodiments of the inventive subject matter may take the form of a computer program product embodied in any tangible or non-transitory medium of expression having computer usable program code embodied in the medium. The described embodiments may be provided as a computer program product, or software, that may include a machine-readable medium having stored thereon instructions, which may be used to program a computing system (or other electronic device(s)) to perform a process according to embodiments, whether presently described or not, since every conceivable variation is not enumerated herein. A machine-readable medium includes any mechanism for storing or transmitting information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read only memory (ROM); random access memory (RAM);

erasable programmable memory (e.g., EPROM and EEPROM); flash memory; or other types of medium suitable for storing electronic instructions. In addition, embodiments may be embodied in an electrical, optical, acoustical or other form of propagated signal (e.g., carrier waves, infrared signals, digital signals, etc.), or wireline, wireless, or other communications medium.

Computer program code for carrying out operations of the embodiments may be written in any combination of one or more programming languages, including an object-oriented programming language such as Java, Smalltalk, C++ or the like and conventional procedural programming languages, such as the “C” programming language or similar programming languages. The program code may execute entirely on a user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN), a personal area network (PAN), or a wide area network (WAN), or the connection may be made to an external computer (e.g., through the Internet using an Internet Service Provider).

The illustrative embodiments may be particularly useful for addressing the affects and after0-affects of SARS-CoV-2 Virus, permutations, variations, and mutations. For example, the virus may be deactivated in treated blood to act as a self-imposed vaccine or one that may be shared with others (i.e., once tested and approved). The illustrative embodiments may be utilized proactively to help patients that are intoxicated, hungover, needing a pick-me-up, or feeling vitamin deficient. The illustrative embodiments may be implemented through an extract, treat, and reinject process (one way) or through a loop or circular system (two way) that is performed on a user in real-time or near real-time. For example, blood of a patient may be treated over an hour to deactive pathogens and help the user feel better. Injection sites may vary (e.g., wrists, arms, legs, neck, etc.). In some embodiments, the ultraviolet lights may also be applied topically for penetration in and through the skin, veins, and tissue of the user. For example, an autologous blood dialysis may be performed to treat the patient, donor blood, or so forth.

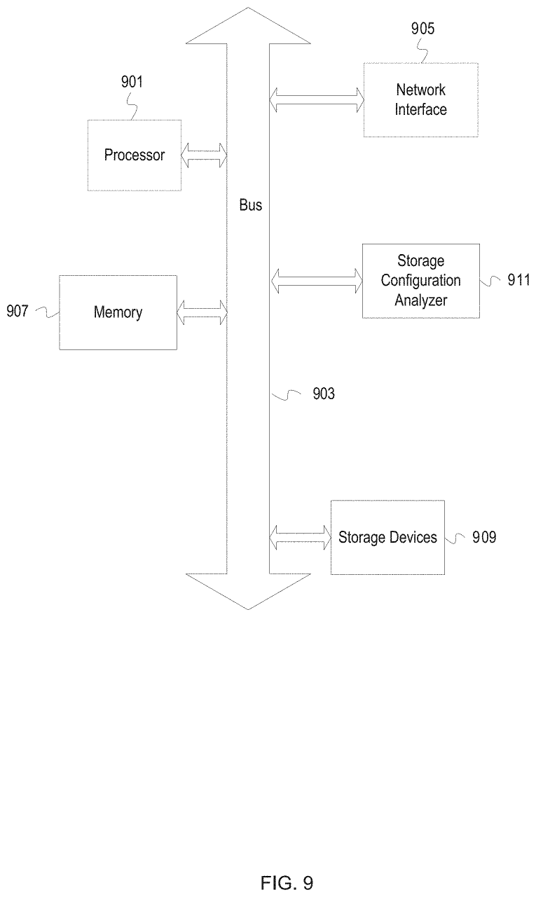

FIG. 9 depicts a computing system 900 in accordance with an illustrative embodiment. For example, the computing system 900 may represent a device, such as one or more of the devices 101 of FIG. 1. The computing system 900 includes a processor unit 901 (possibly including multiple processors, multiple cores, multiple nodes, and/or implementing multi-threading, etc.). The computing system includes memory 907. The memory 907 may be system memory (e.g., one or more of cache, SRAM, DRAM, zero capacitor RAM, Twin Transistor RAM, eDRAM, EDO RAM, DDR RAM, EEPROM, NRAM, RRAM, SONOS, PRAM, etc.) or any one or more of the above already described possible realizations of machine-readable media. The computing system also includes a bus 903 (e.g., PCI, ISA, PCI-Express, HyperTransport®, InfiniBand®, NuBus, etc.), a network interface 905 (e.g., an ATM interface, an Ethernet interface, a Frame Relay interface, SONET interface, wireless interface, etc.), and a storage device(s) 909 (e.g., optical storage, magnetic storage, etc.). The system memory 907 embodies functionality to implement embodiments described above. The system memory 907 may include one or more functionalities that store content, blockchain data, parameters, application, user profiles, and so forth. Code may be implemented in any of the other devices of the computing system 900. Any one of these functionalities may be partially (or entirely) implemented in hardware and/or on the processing unit 901. For example, the functionality may be implemented with an application specific integrated circuit, in logic implemented in the processing unit 901, in a co-processor on a peripheral device or card, etc. Further, realizations may include fewer or additional components not illustrated in FIG. 9 (e.g., video cards, audio cards, additional network interfaces, peripheral devices, etc.). The processor unit 901, the storage device(s) 909, and the network interface 905 are coupled to the bus 903. Although illustrated as being coupled to the bus 903, the memory 907 may be coupled to the processor unit 901.

The features, steps, and components of the illustrative embodiments may be combined in any number of ways and are not limited specifically to those described. For example, the description and figures for FIGS. 1-10 are applicable, combinable, and applicable in various contemplated combinations. In particular, the illustrative embodiments contemplate numerous variations in the smart devices and communications described. The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of embodiments, processes, or methods of the invention. It is understood that any other modifications, substitutions, and/or additions may be made, which are within the intended spirit and scope of the disclosure. For the foregoing, it can be seen that the disclosure accomplishes at least all of the intended objectives.

The previous detailed description is of a small number of embodiments for implementing the invention and is not intended to be limiting in scope. The following claims set forth a number of the embodiments of the invention disclosed with greater particularity.

What is claimed is:

1. A method, comprising:
injecting treated blood into a patient where the treated blood comprises a complex of bodily fluid extracted from the patient and at least one catalyst,
wherein the treated blood has been exposed to at least one emitter.

2. The method of claim 1, wherein the emitter is an ultraviolet (UV) bulb emitting UV-A, UV-B, or UV-C.

3. The method of claim 1, wherein the treated blood is external to the patient's body for no more than 15 minutes.

4. The method of claim 1, wherein the wherein the one or more catalysts include riboflavin.

5. The method of claim 4, wherein the emitter is an ultraviolet (UV) bulb emitting UV-A, UV-B, or UV-C.

6. The method of claim 4, wherein the ratio of bodily fluid to riboflavin is 6 to 1.

7. The method of claim 4, wherein the at least 10 ml of riboflavin is present in the treated blood.

8. The method of claim 6, wherein the at least 10 ml of riboflavin is present in the treated blood.

9. An autologous system for treating blood, comprising:
a first needle connected to tubing for extracting blood from a patient;
a testing module connected to the tubing for testing the blood from the patient for pathogens and conditions;
a catalyst module connected to the testing module adding one or more catalysts to the blood;
a treatment module connected to the catalyst module to activate the catalyst utilizing one or more emissions to generate treated blood;
an additive module connected to the treatment module adding one or more additives to the treated blood;
a second needle connected to the additive module for injecting the treated blood into the patient.

10. The autologous system of claim 9, further comprising:
a second testing module connected to the treatment module for further testing the treated blood for the pathogens and conditions.

11. The autologous system of claim 9, wherein the needle is a light guide for exposing the blood to one or emissions.

12. The autologous system of claim 9, wherein the one or more catalysts include at least riboflavin and wherein the one or more emissions are ultraviolet light.

13. The autologous system of 9, further comprising:
logic controlling the testing module, the treatment module, and the additive module in response to patient data, the pathogens, and the conditions.

14. The autologous system of claim 9, wherein the logic automatically determines the one or more catalysts, the one or more emissions, and the one or more additives to treat the blood.

15. The autologous system of claim 9, wherein the one or more additives are vaccines.

16. The autologous system of claim 9, wherein the treatment module applies the one or more emissions to a plurality of surfaces of the blood.
